(19) United States
(12) Patent Application Publication
(10) Pub. No.: US 2025/0195001 A1
(43) Pub. Date: Jun. 19, 2025

(54) LOWER EXTREMITY VASCULAR LESION EVALUATION DEVICE AND LOWER EXTREMITY VASCULAR LESION EVALUATION METHOD

(71) Applicant: HAMAMATSU PHOTONICS K.K., Hamamatsu-shi, Shizuoka (JP)

(72) Inventors: Naoki SUZUKI, Fushimi-ku, Kyoto-shi, Kyoto (JP); Toshihiko MIZUNO, Hamamatsu-shi, Shizuoka (JP); Takashi WATANABE, Hamamatsu-shi, Shizuoka (JP)

(73) Assignee: HAMAMATSU PHOTONICS K.K., Hamamatsu-shi, Shizuoka (JP)

(21) Appl. No.: 18/845,461

(22) PCT Filed: Feb. 20, 2023
(86) PCT No.: PCT/JP2023/006063

§ 371 Date: Sep. 10, 2024

(30) Foreign Application Priority Data
Mar. 14, 2022 (JP) 2022-038944

(51) Int. Cl. A61B 5/00; A61B 5/1455

(52) U.S. Cl. CPC A61B 5/6829; A61B 5/14552; A61B 5/7246

(57) ABSTRACT

A lower extremity vascular disease evaluation apparatus includes a measurement unit and an evaluation unit. The measurement unit irradiates a foot sole of an evaluation subject with near-infrared light, receives the near-infrared light which is scattered or absorbed by a tissue inside the foot sole, and measures a regional oxygen saturation of the foot sole based on the received light intensity. The evaluation unit evaluates lower extremity vascular disease of the evaluation subject based on a measured value of the regional oxygen saturation of the foot sole.

### TECHNICAL FIELD

The present disclosure relates to an apparatus and a method for evaluating lower extremity vascular disease.

### BACKGROUND ART

Lower extremity vascular disease, which is also called peripheral arterial disease (PAD), is a disease caused by insufficient supply of nutrients and oxygen to a lower extremity due to blood circulatory disorder caused by narrowing and blockage of a blood vessel of the lower extremity, and various disorders appear. The lower extremity vascular disease is caused by arteriosclerosis, and it is considered that the lower extremity vascular disease is more likely to occur when a person has lifestyle disease such as diabetes, dyslipidemia, high blood pressure, smoking, hyperuricemia, chronic kidney disease, obesity, or the like.

Symptoms of the lower extremity vascular disease begin with numbness and coldness of a foot, and progress to ulcer and necrosis via pain in the foot during walking and pain at rest. A patient with the severe lower extremity vascular disease has a poor prognosis, and lower extremity amputation often occurs. It is known that, when the lower extremity of a dialysis patient is amputated, a subsequent survival rate of the dialysis patient is low (for example, a five-year survival rate of 15%). Therefore, early detection and treatment are important for the lower extremity vascular disease.

The detection of the lower extremity vascular disease is performed by confirming symptoms and external appearance as a screening test, and further performing an ankle brachial pressure index (ABI) test. In the ABI test, a ratio of the respective systolic blood pressures of the ankle and the upper arm (a systolic blood pressure of the ankle/a systolic blood pressure of the upper arm) is obtained, and when the above ratio is lower than a certain threshold value, it is determined that there is a suspicion of the lower extremity vascular disease (see Non Patent Document 1).

In the case of the dialysis patient, in a dialysis facility, a foot care is performed, and in addition, a screening test is performed. For the patient who is determined to have a suspicion of the lower extremity vascular disease in the screening test, a non-invasive test (a vascular echography, a magnetic resonance angiography) or an invasive test (a CT by using a contrast agent, an arteriography) is further performed as a detailed examination.

### CITATION LIST

### Non Patent Literature

Non Patent Document 1: Atsuhiro Shimakura, Masanobu Takata, “Vascular Function Tests-CAVI, PWV, ABI”, The Journal of the Japanese Society of Internal Medicine (Nihon Naika Gakkai Zasshi), Volume 102, Issue 2, pp. 335-343, 2013 Feb. 10

### SUMMARY OF INVENTION

### Technical Problem

The ABI test, which is performed as the screening test for the lower extremity vascular disease, is a simple method, but the detection sensitivity of the disease is low, and there are cases in which the lower extremity vascular disease cannot be detected at an early stage. Although testing techniques for the lower extremity vascular disease other than the ABI test are known, none of the techniques is a simple method, and is not suitable for use as the screening test.

An object of an embodiment is to provide a lower extremity vascular disease evaluation apparatus and a lower extremity vascular disease evaluation method which can be suitably used for early detection of lower extremity vascular disease with ease and high sensitivity.

### Solution to Problem

An embodiment of a first aspect is a lower extremity vascular disease evaluation apparatus. The lower extremity vascular disease evaluation apparatus includes a measurement unit for irradiating a foot sole of an evaluation subject with near-infrared light, receiving the near-infrared light scattered or absorbed by a tissue inside the foot sole, and measuring a regional oxygen saturation of the foot sole based on a received light intensity; and an evaluation unit for evaluating lower extremity vascular disease of the evaluation subject based on the regional oxygen saturation of the foot sole.

An embodiment of a second aspect is a lower extremity vascular disease evaluation apparatus. The lower extremity vascular disease evaluation apparatus includes a measurement unit for irradiating a foot sole of an evaluation subject with near-infrared light, receiving the near-infrared light scattered or absorbed by a tissue inside the foot sole, and measuring a regional oxygen saturation of the foot sole based on a received light intensity, and for irradiating an arm or a head of the evaluation subject with the near-infrared light, receiving the near-infrared light scattered or absorbed by a tissue inside the arm or the head, and measuring a regional oxygen saturation of the arm or the head based on a received light intensity; and an evaluation unit for evaluating lower extremity vascular disease of the evaluation subject based on a ratio or a difference between the regional oxygen saturation of the foot sole and the regional oxygen saturation of the arm or the head.

An embodiment of a first aspect is a lower extremity vascular disease evaluation method. The lower extremity vascular disease evaluation method includes a measurement step of irradiating a foot sole of an evaluation subject with near-infrared light, receiving the near-infrared light scattered or absorbed by a tissue inside the foot sole, and measuring a regional oxygen saturation of the foot sole based on a received light intensity; and an evaluation step of acquiring an index for evaluating lower extremity vascular disease of the evaluation subject by comparing the regional oxygen saturation of the foot sole with a threshold value.

An embodiment of a second aspect is a lower extremity vascular disease evaluation method. The lower extremity vascular disease evaluation method includes a first measurement step of irradiating a foot sole of an evaluation subject with near-infrared light, receiving the near-infrared light scattered or absorbed by a tissue inside the foot sole, and measuring a regional oxygen saturation of the foot sole based on a received light intensity; a second measurement step of irradiating an arm or a head of the evaluation subject with the near-infrared light, receiving the near-infrared light scattered or absorbed by a tissue inside the arm or the head, and measuring a regional oxygen saturation of the arm or the head based on a received light intensity; and an evaluation step of acquiring an index for evaluating lower extremity vascular disease of the evaluation subject by comparing a ratio or a difference between the regional oxygen saturation of the foot sole and the regional oxygen saturation of the arm or the head with a threshold value.

### Advantageous Effects of Invention

According to the lower extremity vascular disease evaluation apparatus and the lower extremity vascular disease evaluation method of the embodiments, it is possible to detect the lower extremity vascular disease at an early stage easily and with high sensitivity.

### BRIEF DESCRIPTION OF DRAWINGS

FIG. 1 is a diagram illustrating a configuration of a lower extremity vascular disease evaluation apparatus 1A according to a first embodiment.

FIG. 2 is a diagram illustrating a probe 11.

FIG. 3 is a diagram illustrating a configuration of a lower extremity vascular disease evaluation apparatus 1B according to a second embodiment.

### DESCRIPTION OF EMBODIMENTS

Hereinafter, embodiments of a lower extremity vascular disease evaluation apparatus and a lower extremity vascular disease evaluation method will be described in detail with reference to the accompanying drawings. In the description of the drawings, the same elements will be denoted by the same reference signs, and redundant description will be omitted. The present invention is not limited to these examples, and the Claims, their equivalents, and all the changes within the scope are intended as would fall within the scope of the present invention.

### First Embodiment

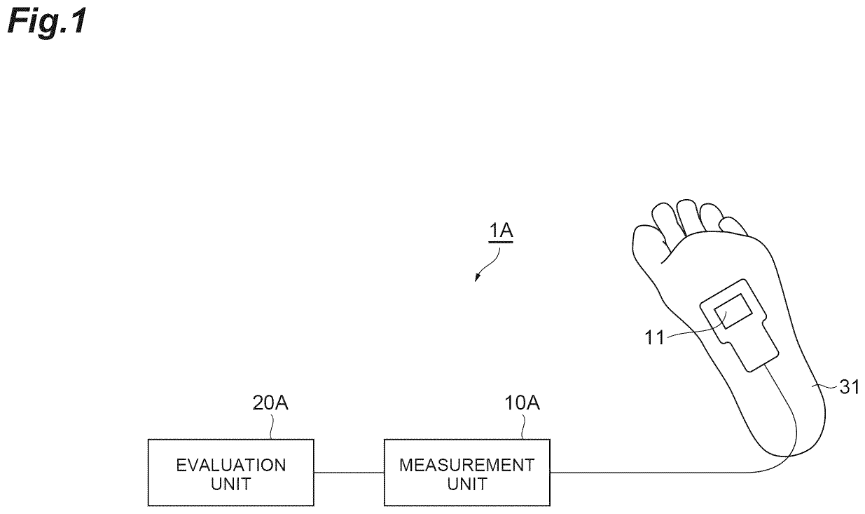

FIG. 1 is a diagram illustrating a configuration of a lower extremity vascular disease evaluation apparatus 1A according to a first embodiment. The lower extremity vascular disease evaluation apparatus 1A includes a measurement unit 10A and an evaluation unit 20A. The measurement unit 10A measures a regional oxygen saturation of a foot sole 31 of an evaluation subject. The evaluation unit 20A evaluates the lower extremity vascular disease of the evaluation subject based on a measured value of the regional oxygen saturation of the foot sole 31. The regional oxygen saturation (regional saturation of oxygen, rSO2) indicates a ratio of the oxygenated hemoglobin in the total hemoglobin. There is a good correlation between the rSO2 and the blood flow.

The measurement unit 10A is used together with a probe 11 provided on the foot sole 31 of the evaluation subject, and can measure the rSO2 of the foot sole 31 by using near infrared spectroscopy (NIRS). A main unit of the measurement unit 10A and the probe 11 are electrically connected by a flexible cable.

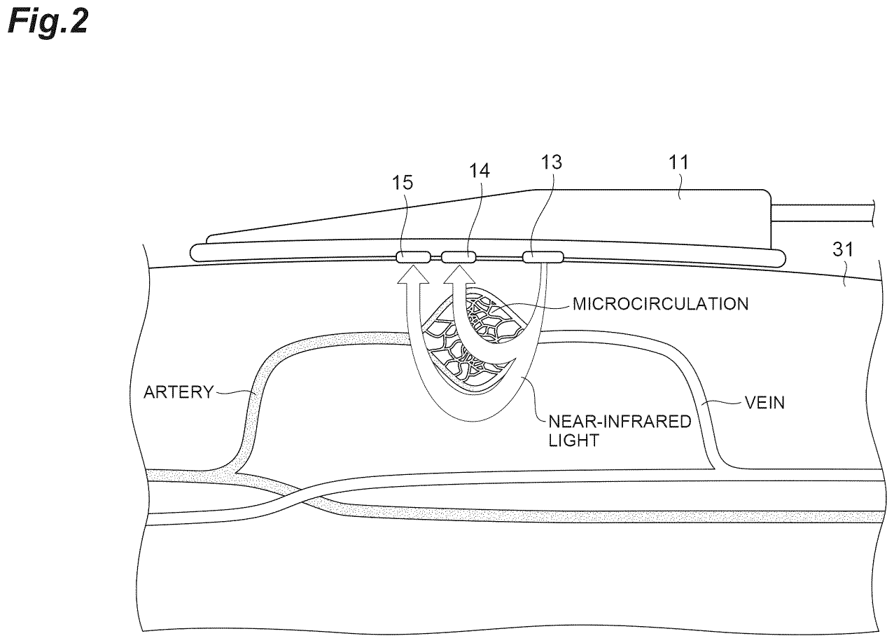

As illustrated in FIG. 2, the probe 11 includes an irradiation unit 13 and detection units 14 and 15 which are spaced apart from each other by a predetermined distance. The distance between the irradiation unit 13 and the detection unit 14 may be set to, for example, 30 mm, and the distance between the irradiation unit 13 and the detection unit 15 may be set to, for example, 40 mm. By setting the above distances respectively to appropriate values, tissue information of a deep portion of the foot sole 31 can be obtained. The main unit of the measurement unit 10A instructs the irradiation unit 13 to emit light, and receives detection results from the detection units 14 and 15.

The probe 11 irradiates the foot sole 31 with near-infrared light (preferably, near-infrared light having a plurality of center wavelengths) from the irradiation unit 13, and receives the near-infrared light after the above near-infrared light is scattered, absorbed, and the like by a tissue inside the foot sole 31 by the detection units 14 and 15. The irradiation unit 13 includes, for example, a light emitting diode as a light source for outputting the near-infrared light, and further, includes a drive circuit for driving the light source.

Each of the detection units 14 and 15 includes, for example, a photodiode as an element for receiving the near-infrared light, and further, includes a circuit for outputting an electrical signal having a value according to an intensity of light received by the element. The measurement unit 10A can measure the rSO2 of the tissue in the foot sole 31 (the tissue in a range of several cm from a surface) based on the detection result of the received light intensity by the detection units 14 and 15. In addition, the near-infrared light in this case refers to light having a center wavelength in a range of 700 nm or more and 2500 nm or less.

The probe 11 may have a configuration including the irradiation unit 13 and one detection unit, and in addition, it is preferable that the prove 11 has a configuration including the irradiation unit 13 and a plurality of detection units. In the case of the configuration including the plurality of detection units, the probe 11 can measure the rSO2 at each of a plurality of portions of the foot sole 31. In this case, any one of the rSO2 measured values obtained for the plurality of portions may be used, or an average value of the rSO2 measured values may be used. Further, in the case in which it is determined that any one measured value of the rSO2 measured values obtained for the plurality of portions is abnormal, the above abnormal value may be excluded, and other measured values may be used.

The measurement unit 10A can easily measure the foot sole rSO2 non-invasively and continuously (or at very short time intervals). The apparatus capable of measuring the rSO2 is commercially available as a product, for example, provided by Hamamatsu Photonics K. K.

The measurement of the rSO2 of the foot sole is preferably performed when the evaluation subject is in the resting state. In the case in which the measured value of the rSO2 of the foot sole changes with time, it is preferable that the measurement is performed over a predetermined period (for example, one minute), and it is also preferable that an average value of the measured values obtained in the above predetermined period is set as the measured value of the foot sole rSO2. Further, a maximum value, a minimum value, or a median value of the measured values obtained in the predetermined period may be set as the measured value of the foot sole rSO2.

The evaluation unit 20A evaluates the lower extremity vascular disease of the evaluation subject based on the measured value of the foot sole rSO2 obtained by the measurement unit 10A. There is a good correlation between the rSO2 and the blood flow, and the lower the measured value of the foot sole rSO2 is, the worse the blood circulation in the tissue inside the foot sole 31 is, and therefore, the lower extremity vascular disease of the evaluation subject can be detected at an early stage based on the measured value of the foot sole rSO2.

The evaluation unit 20A may provide an index for evaluating the lower extremity vascular disease of the evaluation subject by comparing the measured value of the foot sole rSO2 with a threshold value. Further, the evaluation unit 20A may provide the index for evaluating in detail the lower extremity vascular disease of the evaluation subject by setting a plurality of threshold values, and comparing the measured value of the foot sole rSO2 with each of the plurality of threshold values. A doctor or the like can easily evaluate a magnitude of possibility that the evaluation subject has the lower extremity vascular disease by acquiring the index which is described above.

The evaluation unit 20A includes an operation unit for performing an operation including the comparison described above, a storage unit for storing the measured value of the foot sole rSO2, the threshold value, the evaluation result, and the like, and also storing a required program, an input unit for receiving an instruction such as a measurement start, a measurement condition, and the like, and further, a display unit for displaying the measured value of the foot sole rSO2, the evaluation result, and the like. The evaluation unit 20A may be constituted by using a computer, a tablet, or the like.

It is preferable that the display unit of the evaluation unit 20A sequentially displays the measurement result of the foot sole rSO2 by the measurement unit 10A at each time in the measurement period, and it is also preferable that the display unit sequentially displays the average value of the measured values in the predetermined period before the time. Further, it is also preferable that the display unit displays the comparison result between the measured value of the foot sole rSO2 and the threshold value, and displays the degree of the lower extremity vascular disease of the evaluation subject.

It is preferable that, in the case in which the measured value of the foot sole rSO2 is lower than the threshold value, the evaluation unit 20A evaluates the state as a disease worsening state, and displays the evaluation result on the display unit. Further, it is preferable that, in the case in which the measured value of the foot sole rSO2 is equal to or larger than the threshold value, the evaluation unit 20A evaluates the state as a disease improvement state or a continuation suspicion state, and displays the evaluation result on the display unit.

A lower extremity vascular disease evaluation method according to the first embodiment includes a measurement step and an evaluation step. In the measurement step, the foot sole rSO2 of the evaluation subject is measured. The process of the measurement step is performed by using the measurement unit 10A.

In the evaluation step, the lower extremity vascular disease of the evaluation subject is evaluated based on the measured value of the foot sole rSO2. The process of the evaluation step may be performed by using the evaluation unit 20A, or the same process as the process content of the evaluation unit 20A may be performed by a doctor or the like.

Next, results of the measurements performed by the present inventors in order to compare the detection sensitivities of the lower extremity vascular disease respectively by using the evaluation methods of an example and a comparative example will be described. In the example, for each of the evaluation subjects (dialysis patients), the foot sole rSO2 was measured by using the lower extremity vascular disease evaluation method according to the present embodiment described above, within one hour from a start of dialysis, and one minute after pulse waves became stable at a resting supine position. The measurement of the foot sole rSO2 was performed for one minute, and the average of the measured values for one minute was set as the measured value of the foot sole rSO2.

In the comparative example, a ratio of respective systolic blood pressures of an ankle and an upper arm of each of the evaluation subjects (the systolic blood pressure of the ankle/the systolic blood pressure of the upper arm) was measured by using the ABI test according to the ACC/AHA guideline (the guideline of the American College of Cardiology and the American Heart Association).

In each of the example and the comparative example, the measurement was performed for each of 71 lower extremities of the 36 dialysis patients (one of them with right lower extremity amputation) as the evaluation objects. In this case, “one or more below knee arteries are blocked and a blood flow velocity of foot dorsal arteries is not observed” was set as the disease criteria. The above disease criteria are criteria serving as a guide for early detection of the lower extremity vascular disease. As a result of lower extremity arterial echography, 12 extremities out of 71 extremities of the lower extremities satisfied the disease criteria described above.

For each of the example and the comparative example, the detection sensitivity for the above disease criteria was analyzed by using a ROC analysis to obtain a cutoff value and an AUC value. The receiver operating characteristic (ROC) analysis is a statistical analysis method for analyzing the usefulness of the diagnostic method. In the ROC analysis, in a curve (ROC curve) obtained by plotting sensitivity and specificity in the case in which the specific cutoff value is set respectively on the vertical axis and the horizontal axis, and connecting them with a broken line, the closer to 1 the AUC value quantified by an area under the curve (AUC) is, the higher the discrimination ability is.

In the comparative example, the AUC value is 0.527 for the cutoff value of 0.74. On the other hand, in the example, the AUC value is 0.803 for the cutoff value of 33. From the above result, it can be seen that, in early detection of the lower extremity vascular disease, the lower extremity vascular disease evaluation method according to the present embodiment can detect the disease with high sensitivity as compared with the ABI test.

For each of the evaluation objects described above (the 71 lower extremities of the 36 dialysis patients), a foot dorsum rSO2, a fibular ankle rSO2, and a tibial ankle rSO2 were measured, and the detection sensitivity for the above disease criteria was analyzed by using the ROC analysis to obtain the cutoff value and the AUC value. As a result, for the foot dorsum rSO2, the AUC value is 0.594 for the cutoff value of 25. For the fibular ankle rSO2, the AUC value is 0.501 for the cutoff value of 37. For the tibial ankle rSO2, the AUC value is 0.684 for the cutoff value of 42.

The foot sole, the foot dorsum, the fibular ankle, and the tibial ankle are all the ankle and a portion beyond the ankle, and further, it can be seen that the lower extremity vascular disease evaluation method according to the present embodiment in which the foot sole rSO2 is measured can detect the disease at an early stage with high sensitivity as compared with the case of measuring the foot dorsum rSO2, the fibular ankle rSO2, or the tibial ankle rSO2.

Further, the present inventors performed treatment of the dialysis patient by using Rheocarna (registered trademark), which is an adsorption type blood purification device manufactured by Kaneka Medix Corporation, and measured the foot sole rSO2 of the dialysis patient by using the lower extremity vascular disease evaluation method according to the present embodiment in the period of the treatment to confirm the transition of the measured value of the foot sole rSO2 over the course of the treatment.

The Rheocarna is expected to have effects of adsorbing low density lipoprotein cholesterol (LDL cholesterol) by the action of dextran sulfate immobilized by cellulose beads, and adsorbing fibrinogen by the action of L-tryptophan, thereby improving the blood circulation of peripheral arterioles. By continuously performing the treatment by using the Rheocarna, a tendency of improving the disease is confirmed in the external observation, and in addition, a tendency of gradually increasing the measured value of the foot sole rSO2 by using the lower extremity vascular disease evaluation method according to the present embodiment is confirmed.

As described above, it can be seen that the lower extremity vascular disease evaluation apparatus and the lower extremity vascular disease evaluation method according to the present embodiment can detect the lower extremity vascular disease at an early stage easily and with high sensitivity, and can be suitably used as the screening test.

### Second Embodiment

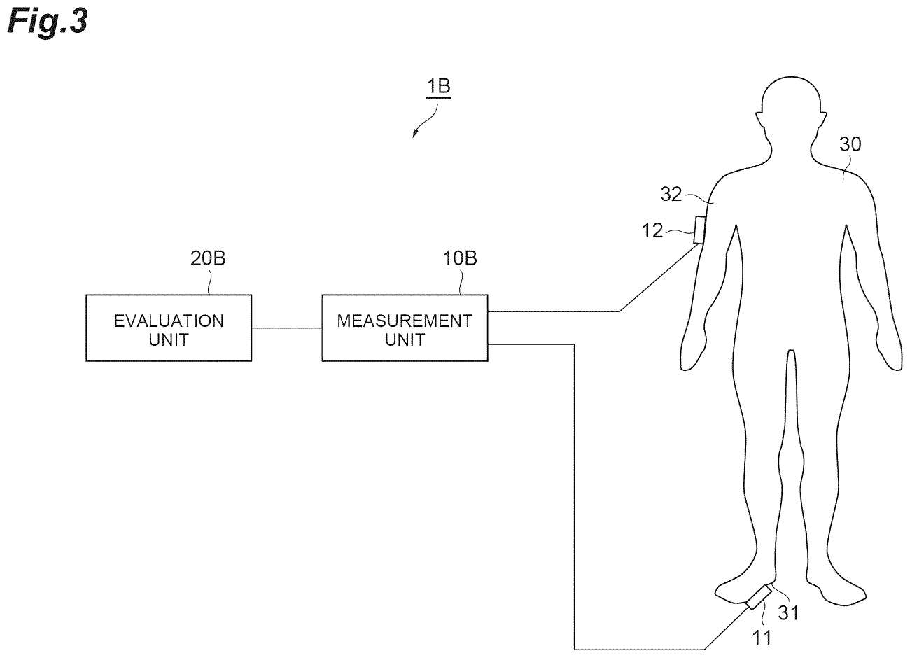

FIG. 3 is a diagram illustrating a configuration of a lower extremity vascular disease evaluation apparatus 1B according to a second embodiment. The lower extremity vascular disease evaluation apparatus 1B includes a measurement unit 10B and an evaluation unit 20B. The measurement unit 10B measures the rSO2 of the foot sole 31 of the evaluation subject 30, and in addition, measures the rSO2 of an arm 32 of the evaluation subject 30. The evaluation unit 20B evaluates the lower extremity vascular disease of the evaluation subject 30 based on the measured value of the rSO2 of the foot sole 31 and the measured value of the rSO2 of the arm 32.

As compared with the measurement unit 10A in the first embodiment, the measurement unit 10B in the second embodiment is different in that the foot sole rSO2 is measured by using the probe 11 provided on the foot sole 31 of the evaluation subject 30, and in addition, the arm rSO2 is measured by using a probe 12 provided on the arm 32 of the evaluation subject 30. The probes 11 and 12 used in the second embodiment have the same configuration as the probe 11 used in the first embodiment.

The measurement of the rSO2 of the arm is also preferably performed when the evaluation subject is in the resting state. In the case in which the measured value of the rSO2 of the arm changes with time, it is preferable that the measurement is performed over a predetermined period (for example, one minute), and it is also preferable that the average value of the measured values obtained in the above predetermined period is set as the measured value of the arm rSO2. Further, the maximum value, the minimum value, or the median value of the measured values obtained in the predetermined period may be set as the measured value of the arm rSO2.

The rSO2 can be stably measured at a portion of the arm where there are muscles, and therefore, it is preferable to measure the rSO2 on an outer side of an upper arm, and further, it is also preferable to measure the rSO2 on a forearm (in particular, on an outer side near an elbow).

The evaluation unit 20B evaluates the lower extremity vascular disease of the evaluation subject 30 based on a ratio or a difference between the measured value of the foot sole rSO2 and the measured value of the arm rSO2 obtained by the measurement unit 10B. The measured value of the foot sole rSO2 indicates the degree of the blood flow in the tissue in the foot sole 31, and the measured value of the arm rSO2 indicates the degree of the blood flow in the tissue in the arm 32. It is considered that the measured value of the foot sole rSO2 decreases with the progress of the lower extremity vascular disease, and on the other hand, the measured value of the arm rSO2 does not change regardless of the degree of progress of the lower extremity vascular disease, and therefore, the lower extremity vascular disease of the evaluation subject 30 can be detected at an early stage with high accuracy based on the ratio or the difference between the measured value of the foot sole rSO2 and the measured value of the arm rSO2.

The evaluation unit 20B may provide the index for evaluating the lower extremity vascular disease of the evaluation subject 30 by comparing the ratio or the difference between the measured value of the foot sole rSO2 and the measured value of the arm rSO2 with the threshold value. Further, the evaluation unit 20B may provide the index for evaluating in detail the lower extremity vascular disease of the evaluation subject 30 by setting the plurality of threshold values, and comparing the ratio or the difference between the measured value of the foot sole rSO2 and the measured value of the arm rSO2 with each of the plurality of threshold values. A doctor or the like can easily evaluate a magnitude of possibility that the evaluation subject 30 has the lower extremity vascular disease by acquiring the index which is described above.

The evaluation unit 20B includes an operation unit for performing an operation including the comparison described above, a storage unit for storing the measured value of the foot sole rSO2, the measured value of the arm rSO2, the ratio or the difference between them, the threshold value, the evaluation result, and the like, and also storing a required program, an input unit for receiving an instruction such as a measurement start, a measurement condition, and the like, and further, a display unit for displaying the measured value of the foot sole rSO2, the measured value of the arm rSO2, the evaluation result, and the like. The evaluation unit 20B may be constituted by using a computer, a tablet, or the like.

It is preferable that the display unit of the evaluation unit 20B sequentially displays the measurement result of each of the foot sole rSO2 and the arm rSO2 by the measurement unit 10B at each time in the measurement period, and it is also preferable that the display unit sequentially displays the average value of the measured values in the predetermined period before the time. Further, it is also preferable that the display unit displays the comparison result between the ratio or the difference between the measured value of the foot sole rSO2 and the measured value of the arm rSO2 and the threshold value, and displays the degree of the lower extremity vascular disease of the evaluation subject.

It is preferable that, in the case in which the ratio or the difference between the measured value of the foot sole rSO2 and the measured value of the arm rSO2 is lower than the threshold value, the evaluation unit 20B evaluates the state as a disease worsening state, and displays the evaluation result on the display unit. Further, it is preferable that, in the case in which the ratio or the difference between the measured value of the foot sole rSO2 and the measured value of the arm rSO2 is equal to or larger than the threshold value, the evaluation unit 20B evaluates the state as a disease improvement state or a continuation suspicion state, and display the evaluation result on the display unit.

A lower extremity vascular disease evaluation method according to the second embodiment includes a first measurement step, a second measurement step, and an evaluation step. In the first measurement step, the rSO2 of the foot sole 31 of the evaluation subject 30 is measured. In the second measurement step, the rSO2 of the arm 32 of the evaluation subject 30 is measured. The respective processes of the first measurement step and the second measurement step are performed by using the measurement unit 10B. The first measurement step and the second measurement step may be performed at different times, and in addition, it is preferable that the above steps are performed in a common period.

In the evaluation step, the lower extremity vascular disease of the evaluation subject 30 is evaluated based on the ratio or the difference between the measured value of the foot sole rSO2 and the measured value of the arm rSO2. The process of the evaluation step may be performed by using the evaluation unit 20B, or the same process as the process content of the evaluation unit 20B may be performed by a doctor or the like.

In general, the measurement of the rSO2 by using the NIRS is considered to be insufficient in reproducibility. However, in the present embodiment, the evaluation of the lower extremity vascular disease is performed based on the ratio or the difference between the measured value of the foot sole rSO2 and the measured value of the arm rSO2, and thus, it is possible to detect the lower extremity vascular disease at an early stage with stability and high accuracy.

In addition, the measurement unit 10B may measure the rSO2 of a head (in particular, a forehead) instead of the arm 32, and the evaluation unit 20B may evaluate the lower extremity vascular disease of the evaluation subject based on the ratio or the difference between the measured value of the foot sole rSO2 and the measured value of the head rSO2. In this case also, it is possible to detect the lower extremity vascular disease at an early stage with stability and high accuracy.

The lower extremity vascular disease evaluation apparatus and the lower extremity vascular disease evaluation method are not limited to the embodiments and configuration examples described above, and various modifications are possible.

The lower extremity vascular disease evaluation apparatus of the first aspect according to the above embodiment includes a measurement unit for irradiating a foot sole of an evaluation subject with near-infrared light, receiving the near-infrared light scattered or absorbed by a tissue inside the foot sole, and measuring a regional oxygen saturation of the foot sole based on a received light intensity; and an evaluation unit for evaluating lower extremity vascular disease of the evaluation subject based on the regional oxygen saturation of the foot sole.

The lower extremity vascular disease evaluation apparatus of the second aspect according to the above embodiment includes a measurement unit for irradiating a foot sole of an evaluation subject with near-infrared light, receiving the near-infrared light scattered or absorbed by a tissue inside the foot sole, and measuring a regional oxygen saturation of the foot sole based on a received light intensity, and for irradiating an arm or a head of the evaluation subject with the near-infrared light, receiving the near-infrared light scattered or absorbed by a tissue inside the arm or the head, and measuring a regional oxygen saturation of the arm or the head based on a received light intensity; and an evaluation unit for evaluating lower extremity vascular disease of the evaluation subject based on a ratio or a difference between the regional oxygen saturation of the foot sole and the regional oxygen saturation of the arm or the head.

In the lower extremity vascular disease evaluation apparatus of the second aspect, the measurement unit may perform a measurement of the regional oxygen saturation of the foot sole of the evaluation subject and a measurement of the regional oxygen saturation of the arm or the head of the evaluation subject in a common period.

The lower extremity vascular disease evaluation method of the first aspect according to the above embodiment includes a measurement step of irradiating a foot sole of an evaluation subject with near-infrared light, receiving the near-infrared light scattered or absorbed by a tissue inside the foot sole, and measuring a regional oxygen saturation of the foot sole based on a received light intensity; and an evaluation step of acquiring an index for evaluating lower extremity vascular disease of the evaluation subject by comparing the regional oxygen saturation of the foot sole with a threshold value.

The lower extremity vascular disease evaluation method of the second aspect according to the above embodiment includes a first measurement step of irradiating a foot sole of an evaluation subject with near-infrared light, receiving the near-infrared light scattered or absorbed by a tissue inside the foot sole, and measuring a regional oxygen saturation of the foot sole based on a received light intensity; a second measurement step of irradiating an arm or a head of the evaluation subject with the near-infrared light, receiving the near-infrared light scattered or absorbed by a tissue inside the arm or the head, and measuring a regional oxygen saturation of the arm or the head based on a received light intensity; and an evaluation step of acquiring an index for evaluating lower extremity vascular disease of the evaluation subject by comparing a ratio or a difference between the regional oxygen saturation of the foot sole and the regional oxygen saturation of the arm or the head with a threshold value.

In the lower extremity vascular disease evaluation method of the second aspect, the first measurement step and the second measurement step may be performed in a common period.

### INDUSTRIAL APPLICABILITY

The embodiments can be used as a lower extremity vascular disease evaluation apparatus and a lower extremity vascular disease evaluation method which can be suitably used for early detection of lower extremity vascular disease with ease and high sensitivity.

### REFERENCE SIGNS LIST

1A, 1B—lower extremity vascular disease evaluation apparatus, 30—evaluation subject, 31—foot sole, 32—arm, 10A, 10B—measurement unit, 11, 12—probe, 13—irradiation unit, 14, 15—detection unit, 20A, 20B—evaluation unit.

1. A lower extremity vascular disease evaluation apparatus comprising:
a measurement unit configured to irradiate a foot sole of an evaluation subject with near-infrared light, receive the near-infrared light scattered or absorbed by a tissue inside the foot sole, and measure a regional oxygen saturation of the foot sole based on a received light intensity; and
an evaluation unit configured to evaluate lower extremity vascular disease of the evaluation subject based on the regional oxygen saturation of the foot sole.

2. A lower extremity vascular disease evaluation apparatus comprising:
a measurement unit configured to irradiate a foot sole of an evaluation subject with near-infrared light, receive the near-infrared light scattered or absorbed by a tissue inside the foot sole, and measure a regional oxygen saturation of the foot sole based on a received light intensity, and configured to irradiate an arm or a head of the evaluation subject with the near-infrared light, receive the near-infrared light scattered or absorbed by a tissue inside the arm or the head, and measure a regional oxygen saturation of the arm or the head based on a received light intensity; and
an evaluation unit configured to evaluate lower extremity vascular disease of the evaluation subject based on a ratio or a difference between the regional oxygen saturation of the foot sole and the regional oxygen saturation of the arm or the head.

3. The lower extremity vascular disease evaluation apparatus according to claim 2, wherein the measurement unit is configured to perform a measurement of the regional oxygen saturation of the foot sole of the evaluation subject and a measurement of the regional oxygen saturation of the arm or the head of the evaluation subject in a common period.

4. A lower extremity vascular disease evaluation method comprising:
performing a measurement of irradiating a foot sole of an evaluation subject with near-infrared light, receiving the near-infrared light scattered or absorbed by a tissue inside the foot sole, and measuring a regional oxygen saturation of the foot sole based on a received light intensity; and
performing an evaluation of acquiring an index for evaluating lower extremity vascular disease of the evaluation subject by comparing the regional oxygen saturation of the foot sole with a threshold value.

5. A lower extremity vascular disease evaluation method comprising:
performing a first measurement of irradiating a foot sole of an evaluation subject with near-infrared light, receiving the near-infrared light scattered or absorbed by a tissue inside the foot sole, and measuring a regional oxygen saturation of the foot sole based on a received light intensity;
performing a second measurement of irradiating an arm or a head of the evaluation subject with the near-infrared light, receiving the near-infrared light scattered or absorbed by a tissue inside the arm or the head, and measuring a regional oxygen saturation of the arm or the head based on a received light intensity; and
performing an evaluation of acquiring an index for evaluating lower extremity vascular disease of the evaluation subject by comparing a ratio or a difference between the regional oxygen saturation of the foot sole and the regional oxygen saturation of the arm or the head with a threshold value.

6. The lower extremity vascular disease evaluation method according to claim 5, wherein the first measurement and the second measurement are performed in a common period.
